(19) United States
(12) Plant Patent Application Publication
(10) Pub. No.: US 2013/0061359 P1
(43) Pub. Date: Mar. 7, 2013

(54) Rosa hybrida shrub named 'AUSrapper'

(50) ;

(71) Applicant: David C.H. Austin, Wolverhampton (GB)

(72) Inventor: David C.H. Austin, Wolverhampton (GB)

(21) Appl. No.: 13/506,561

(22) Filed: Apr. 27, 2012

(30) Foreign Application Priority Data
Sep. 7, 2011 (EP) 2011/2134

(51) Int. Cl. A01H 5/00

(52) U.S. Cl. PLT/105

(57) ABSTRACT

A variety of rose plant of the shrub class, named ‘AUSrapper’. The ‘AUSrapper’ rose is a plant having beautiful full-petalled, cup-shaped blooms, having at the center of each bloom a button eye of small inward folding petals, an opening color that is a soft creamy apricot, which quickly fades to cream and then to pure white, and a fragrance that is pure myrrh and of medium strength. The petals of the flower drop very cleanly and it forms a most attractive, bushy, but relatively upright shrub that is extremely healthy.

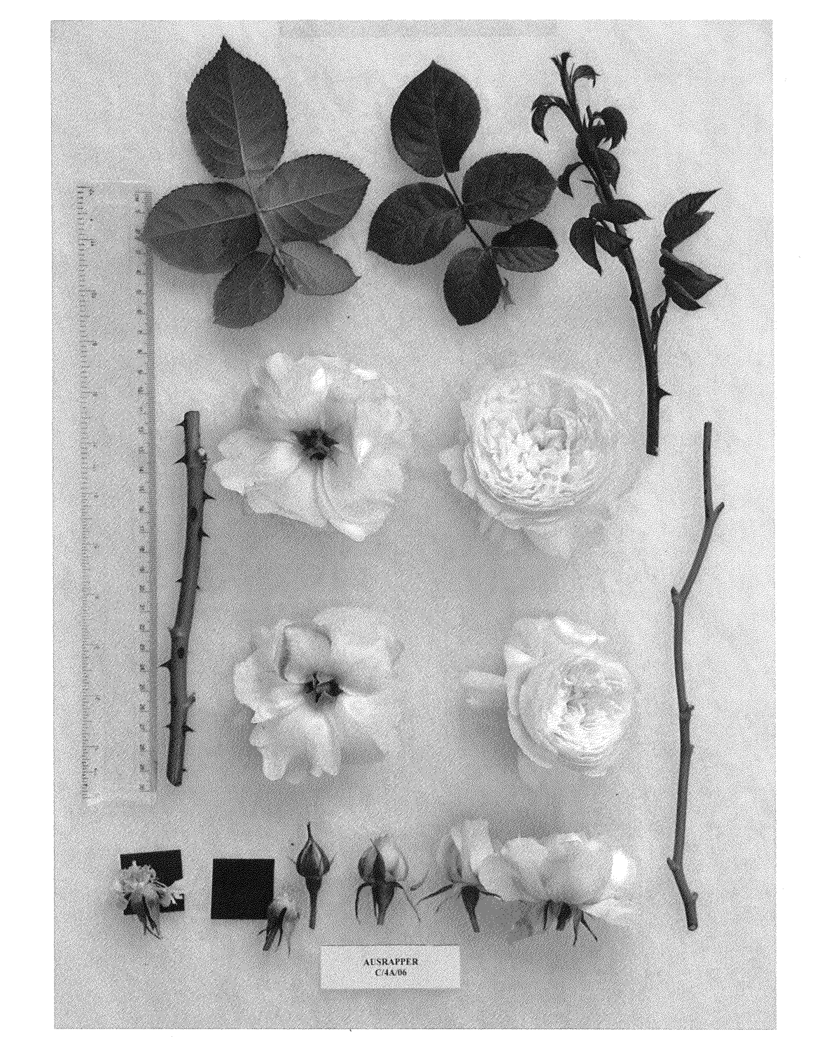

### CROSS-REFERENCE TO RELATED APPLICATIONS

The current application claims priority to a European Community Plant Variety Application No. 2011/2134, filed Sep. 7, 2011, the disclosure of which is incorporated herein by reference.

### CLASSIFICATION

The present invention relates to a new Rosa hybrida shrub.

### VARIETY DENOMINATION

The new plant has the varietal denomination ‘AUSrapper.’

### ORIGIN OF INVENTION

This invention relates to a new and distinct variety of shrub rose plant named ‘AUSrapper’, which was originated from the cross-pollination of the two ‘unnamed’ seedlings of Rosa hybrida.

### BACKGROUND OF THE INVENTION

The primary objective of this breeding was to produce a variety of rose with beautiful full-petalled, cup-shaped blooms, having at the center of each bloom a button eye of small inward folding petals, an opening color that is a soft creamy apricot, which quickly fades to cream and then to pure white, and a fragrance that is pure myrrh and of medium strength. The petals of the flower drop very cleanly and it forms a most attractive, bushy, but relatively upright shrub that is extremely healthy.

### SUMMARY OF THE INVENTION

Among the features which distinguish the new variety from others presently available and commercial rose cultivars known to the inventor is the following combination of characteristics: cup-shaped blooms starting a soft creamy apricot color then quickly fading to cream then white, a many-petalled blooms with a button eye, and a bushy but relatively upright shrub.

Asexual reproduction of the new variety by budding as performed outdoors at Bowling Green Lane in Albrighton, Britain shows that the foregoing and other distinguishing characteristics come true to form and are established and transmitted through succeeding asexual propagations. ‘AUSrapper’ may be asexually propagated by budding. The budding successfully occurred on the plant/rootstock Rosa cv. ‘Dr. Huey’ in the United States, and Rosa dumetorum cv. Laxa’ for the observed plants in the UK.

### COMPARISON WITH PARENTS

The seed and pollen parents of ‘AUSrapper’ are two unnamed seedlings. The new rose may be distinguished from its seed parent, by the following combination of characteristics: the new variety is healthy while the parent has poor growth and is disease prone. The new rose may be distinguished from its pollen parent, by the following combination of characteristics: the new variety has apricot colored flower, while the parent has pink cupped flowers with fewer petals.

### COMPARISON WITH THE CLOSEST COMMERCIALLY AVAILABLE CULTIVAR

The closest commercially available cultivars to the new variety are the ‘AUSprior’ (U.S. Plant Pat. No. 19,465) and ‘AUSrelate’ (U.S. Plant Pat. No. 18,702). The new rose may be distinguished from the ‘AUSprior’ variety by the following combination of characteristics: the ‘AUSrapper’ is bushy and upright reaching 1.2 m, whereas in ‘AUSprior’ has a more arching growth habit and is taller (1.35 m) and will make a climber of up to 2.4 m. ‘AUSrapper’ also has buds that are red and green, while ‘AUSprior’ has buds that show pale yellow-green with red specks. Finally, ‘AUSrapper’ has a more domed shape than ‘AUSrelate’, and ‘AUSrelate’ has blooms that are more apricot than the purer white blooms of ‘AUSrapper’.

### BRIEF DESCRIPTION OF ILLUSTRATION

The accompanying photograph illustrates the new variety and shows the flowering thereof from bud to full bloom depicted in color as nearly correct as it is possible to make in a color illustration of the character. Throughout this specification, color references and/or values are based upon the Colour Chart of The Royal Horticultural Society (2001) except where common terms of color definition are employed.

### DESCRIPTION OF THE NEW VARIETY

Observations of budded ‘AUSrapper’ specimens were made at the nursery at David Austin Roses Limited in Albrighton, Wolverhampton, United Kingdom. The following description is of 1 year-old rose plants of the new variety grown outdoors in Albrighton, Britain in the month of June. Phenotypic expression may vary with environmental, cultural and climatic conditions, as well as differences in conditions of light and soil.
Flower characteristics:
   Blooming habit.—Recurrent. The number of blooms per plant during the growing season is profuse, but there are too many to count.
   Bud.—Size: About 2.7 cm long and 1.6 cm in diameter when the petals start to unfurl. Form: The bud form is short and globular. Color: When sepals first divide, the bud color is red RHS 60A & green RHS 145C. When half blown, the upper sides of the petals are RHS N155D and the lower sides of the petals are RHS N155D. Sepals: Color: Upper Surface: green RHS 147C. Lower Surface: green RHS 144A with some RHS 60B. Length: 2.7 cm. Width: 7 mm. Shape: Lanceolate. Surface texture: Upper Surface: Downy. Lower Surface: Glandular. Number: There are 3 normally appendaged sepals, and 2 unappendaged sepals with hairy edges. Receptacles: Color: green RHS 144A. Shape: funnel. Size: medium, about 1 cm×1 cm. Surface: Smooth. Peduncle: Length: short, averaging about 5 cm. Surface: Glandular. Color: light green RHS 144A. Strength: Strong.
   Blooms.—Size: Medium, average open diameter is about 9 cm. Borne: Several together in clusters of 4-14 blooms having flat shape. Stems: Strength: Strong, average length is about 40 cm. Diameter: 5 mm. Form: When first open, bloom is cupped. Permanence of bloom: outer petal edges curl slightly.
   Petalage.—Number of petals under normal conditions: 115. Color: The upper sides of the petals are white/near white RHS 155B. The reverse sides of the petals are white/near white RHS 155B, and the based of the petals has RHS 155B. Variegations: N/A. Discoloration: The general tonality at the end of the first day is white RHS 155B. Fragrance: Moderate. Character of fragrance: myrrh.
   Petals.—Texture: Smooth. Size: Width: 2.8 cm. Length: 4.5 cm. Surface: Smooth. Shape: Obovate. Margin: Entire with some petals notched. Apex Shape: Weak undulations. Base Shape: Pointed. Form: Incurved. Arrangement: Imbricated. Petaloids: Number: 5. Color: Anther/Filament: RHS 10A, Petal: RHS 155B. Length: Anther: 2 mm, Filament: 6 mm, Petal: 13 mm. Persistence: Petals hang on and dry. Lastingness: On the plant: About 5 days. As a cut flower: Not tested.
   Reproductive parts.—Stamens: Number: 45. Length: 8 mm. Anthers: Length: 2 mm. Color: RHS 12A. Arrangement: mixed with petaloids. Filaments: Color: Yellow RHS 12A. Length: 7 mm. Pollen: Color: gold yellow RHS 17B. Pistils: Number: 20. Length: 3.9 mm. Styles: Color: RHS 2D. Length: 3.5 mm. Stigmas: Color: Yellow RHS 12A. Length: 0.4 mm. Hips: None Observed.
Plant characteristics:
   Plant form.—Shrub.
   Plant growth.—Upright.
   Age to maturity.—Two years.
   Mature plant.—Height: 120 cm. Width: 90 cm.
   Rootstock.—Rosa cv. ‘Dr. Huey’ in the United States, Rosa dumetorum cv. ‘Laxa’ for the observed plants in the UK.
   Foliage.—Number: Leaflets on normal mid-stem leaves is: 5. Size: Medium, about 9.6 cm long×8.7 cm wide. Quantity: Normal. Number of leaves per flowering stem is: 15. Color: New foliage: Upper side: dark green RHS 135A. Lower side: medium green RHS 139B. Old foliage: Upper side: dark green RHS 135A. Lower side: medium green RHS 139B.
   Leaflets.—Size: About 4.25 cm long×3 cm wide. Shape: Pointed oval. Base Shape: Rounded. Apex Shape: Cuspidate. Texture: Leathery. Edge: Serrated. Serrations: Single and small. Petiole: Color: RHS 139B. Length: 5 cm. Width: 1 mm. Surface Texture: With prickles (as rachis). Petiole Rachis: Color: green RHS 139B. Underside: with prickles. Stipules: Length: 2 cm, smooth. Color: RHS 139C. Auricle: Shape: Lanceolate. Length: 8 mm. Width: 2 mm. Color: RHS N138B. Vein Color: RHS 135A. Venation Pattern: Reticulate.
   Wood.—New Wood: Color: violet-red RHS 178A. Bark: Smooth. Old Wood: Color: light-green RHS 146B. Bark: Smooth.
   Stems.—Mature Stem: Length: 40 cm. Diameter: 6 mm. Internode Distance: 8-10 cm. The above measurements are all variable, depending on growing conditions in a season. Stem Pubescence Present: No.
   Prickles.—Quantity: On main canes from base: Ordinary. Number per stem length: 10 per 10 cm. On laterals from main canes: Few. Number per stem length: 4 per 10 cm. Form: Deep concave. Length: Long prickles: 10 mm×8 mm. Short prickles: 4 mm×1 mm. Color when young: Dark red RHS 178A. Color when mature: Brown RHS 173A.
   Small prickles.—Quantity: Main stalk: Few Number per stem length: 15 per stem. Laterals: None Number per stem length: N/A. Color: When young: Red RHS 178A. When mature: Brown RHS 173A.
   Disease resistance.—Mildew: Resistant. Blackspot: Resistant. Rust: Resistant.
   Pest resistance.—Not tested.
   Winter hardiness.—Not tested.
   Growing conditions.—Normal.

What is claimed is:

1. A new and distinct variety of rose plant of the shrub class suitable for repeat flower production, substantially as herein shown and described.
